(19) United States
(12) Patent Application Publication
(10) Pub. No.: US 2008/0161245 A1
(43) Pub. Date: Jul. 3, 2008

(54) Protein-Binding Anthracycline Peptide Derivatives and Drugs Containing Them

(75) Inventors: Felix Kratz, Ihringen (DE); Da-Eun Chung, Dreieich-Sprendlingen (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: KTB Tumorforschungsgesellschaft mbH, Freiburg im Breisgau (DE)

(21) Appl. No.: 11/885,165

(22) PCT Filed: Feb. 22, 2006
(86) PCT No.: PCT/EP06/01623

§ 371 Date: Mar. 12, 2008

(30) Foreign Application Priority Data
Feb. 28, 2005 (DE) 10 2005 009 084.2

(51) Int. Cl. A61K 38/08; C07K 7/06; A61P 35/04

(52) U.S. Cl. 514/16; 530/328; 530/329; 514/17

(57) ABSTRACT

The invention pertains to low-molecular anthracycline-peptide derivatives with PSA-cleavable peptide sequences, which contain a protein-binding group.

### FIELD OF THE INVENTION

The invention pertains to low-molecular anthracycline peptide derivatives which can be cleaved by the prostate-specific antigen (PSA), to their production, and to their use.

### BACKGROUND OF THE INVENTION

Anthracyclines are a group of widely used antineoplastic active agents such as doxorubicin, daunorubicin, and epirubicin, which are used to treat various cancer diseases. The chemotherapeutic treatment of malignant diseases with anthracyclines, however, is associated with side effects as a result of the limited therapeutic range of these active ingredients (Dorr R. T., Von Hoff D. D.: “Cancer Chemotherapy Handbook”, 2^{nd} edition, Appleton and Lange, Norwalk, 1994; Myers C. E., Chabner, B. A.: Anthracyclines. In: “Cancer Chemotherapy—Principles and Practice”, Lippincott, Philadelphia; Chabner, B. A., Collins, J. M. eds., 1990, pp. 356-381). It is known that it has been possible to use certain prodrugs to achieve effective transport of bound active ingredients into affected tissue and also to achieve an efficient and highly specific release of the active ingredient at the target location as a result of certain biochemical and physiological features of the malignant tissue. To improve the side-effect profile and the efficacy of anthracyclines or anthracycline derivatives, protein-binding formulations have been developed, which bind in vivo to endogenous serum proteins, especially albumin, and thus provide macromolecular transport forms of the active ingredients (Kratz et al., J. Med. Chem., 2002, 45, 5523; Mansour et al., Cancer Res. 2003, 63, 4062).

PSA, furthermore, has been identified as a protease in malignant tumors (Levesque, M., Yu, H., D'Costa, M., & Diamandis, E., J. Clin. Lab. Anal., 1995, 9, 123-128). High concentrations of PSA can be detected especially in breast and prostate tissue.

PSA (molecular weight ˜33 kDa) belongs to the protein family of the kallikreins and as a serine protease shows a substrate specificity similar to that of chymotrypsin. The gel-forming proteins semenogelin I and II of seminal fluid are the main substrate for PSA. PSA is synthesized and secreted by prostate gland cells as a proenzyme. Other cells of the body secrete only very small amounts of PSA (Yousef, G. M., Diamandis, E. P.: Endocr. Rev., 2001, 22, 184-204).

Large amounts of PSA are expressed In cases of metastasizing prostate carcinoma, so that the local concentrations have high values in the mg/mL range. PSA is activated in the extracellular space and exerts an enzymatic effect there. In blood plasma, however, PSA binds strongly to α₁-antichymotrypsin and α₂-macroglobulin and as a result has no enzymatic activity. For these reasons, PSA, as a tumor-associated protease, is a highly suitable candidate for the prodrug approach to the effective treatment of PSA-positive prostate tumors.

### SUMMARY OF THE INVENTION

The object underlying the invention is to create derivatives of anthracyclines which, after intravenous administration, bind covalently to circulating albumin and are cleaved by PSA in the tumor tissue to release the active ingredient.

This object is accomplished according to the invention by low-molecular anthracycline-peptide derivatives of the general formula I:

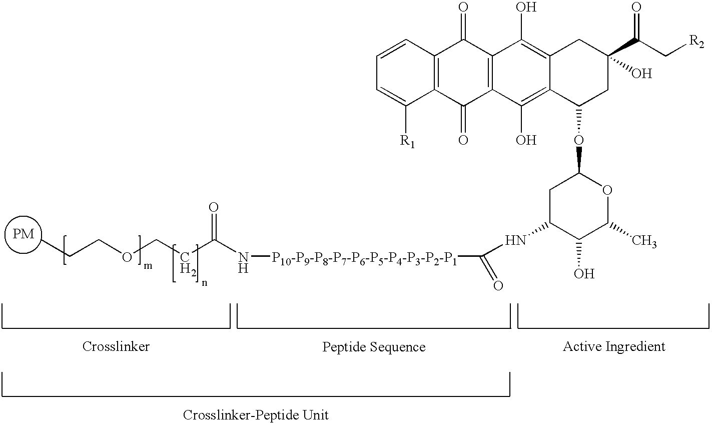

where

R₁═H, OCH₃, or OH;

R₂═H or OH;

m=0-5;

n=0-6;

P₁-P₁₀ is a peptide sequence consisting of L- and/or D-amino acids; and

PM is a protein-binding group.

The anthracycline component of the anthracycline-peptide derivative according to formula I is doxorubicin, daunorubicin, 4′-epirubicin, idarubicin, or carubicin.

The anthracycline component of the anthracycline-peptide derivative according to formula I is doxorubicin.

The PM of the anthracycline-peptide derivative according to formula I is a maleinimide group, a 2-dithiopyridyl group, a halogen acetamide group, a halogen acetate group, a disulfide group, an acrylic acid ester group, a monoalkyl maleic acid ester group, a monoalkyl maleaminic acid amide group, a N-hydroxysuccinimidyl ester group, an isothiocyanate group, or an aziridine group, which can optionally be substituted.

The PM of the anthracycline-peptide derivative according to formula I is a maleinimide group, which can optionally be substituted.

The anthracycline-peptide derivative according to formula I is such that n<2 and m=2-5, or n=4 and m=0.

The P₁ in the peptide sequence (P₁-P₁₀) in the anthracycline-peptide derivative according to formula I is Arg, His, Met, Ser, Tyr, Thr, Phe, Gly, Gln, or Lys.

The P₁ in the peptide sequence (P₁-P₁₀) in the anthracycline-peptide derivative according to formula I is Arg.

The P₁, P₂, P₃, P₄, P₅, and P₆ in the peptide sequence of the anthracycline-peptide derivative according to formula I are the same or different and stand for Ser, Tyr, Thr, Asn, Gln, Gly, or Phe.

The P₁, P₂, P₃, P₄, P₅, and P₆ in the peptide sequence of the anthracycline-peptide derivative according to formula I are the same or different and stand for Ser or Tyr.

The peptide sequence P₁-P₁₀ in the anthracycline-peptide derivative according to formula I comprises six, seven, or eight amino acids.

The peptide sequence of the anthracycline-peptide derivative according to formula I is Arg-Ser-Ser-Tyr-Tyr-Ser-Arg, Arg-Arg-Ser-Ser-Tyr-Tyr-Ser-Gly, Ser-Ser-Tyr-Tyr-Ser-Gly, Asn-Ser-Ser-Tyr-Phe-Gln, Arg-Ser-Ser-Tyr-Tyr-Gln-Arg, or Arg-Ser-Ser-Tyr-Tyr-Tyr-Arg.

The peptide sequence of the anthracycline-peptide derivative according to formula I is Arg-Ser-Ser-Tyr-Tyr-Ser-Arg.

A process for the production of a doxorubicin-peptide derivative according to formula I, the process comprising reacting doxorubicin with a peptide derivative of general formula II:

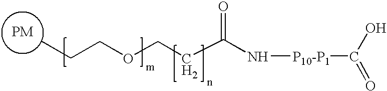

wherein

m=0-5;

n=0-6;

P₁-P₁₀ is a peptide sequence consisting of L- and/or D-amino acids; and

PM is a protein-binding group, in the presence of carboxylic acid activation reagents.

The PM in the above process is a maleinimide group.

A process for the production of doxorubicin-peptide derivatives, said process comprising reacting doxorubicin with a peptide of general formula III:

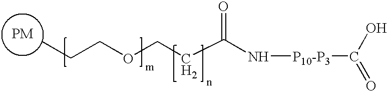

wherein

m=0-5;

n=0-6;

P₃-P₁₀ is a peptide sequence, consisting of L- and/or D-amino acids; and

PM is a protein-binding group, in the presence of carboxylic acid activation reagents.

The PM in any of the above processes is a maleinimide group.

The carboxylic acid activation reagent in any of the above processes is selected from N,N′-dicyclohexylcarbodiimide, N,N′-diisopropylcarbodiimide, N-[(dimethylamino)-1H-1,2,3-triazolo[4,5-b]pyridino-1-ylmethylene]-N-methylmethanaminium hexafluorophosphate (HATU), or (benzotriazol-1-yloxy)tris(dimethylamino)phosphonium hexafluorophosphate, preferably N,N′-diisopropylcarbodiimide.

A composition comprising the anthracycline-peptide derivative of formula I, optionally together with pharmaceutical auxiliary substances and/or solvents.

A method for the treatment of cancer diseases comprising administering to a subject in need of treatment of cancer disease a composition comprising the anthracycline-peptide derivative of formula I.

A process for the production of a composition, the process comprising converting a compound of formula I into a therapeutically compatible solution.

The inventive compounds are constructed from an anthracycline active ingredient, a peptide spacer, and a heterobifunctional crosslinker. This structure is explained in greater detail below.

### BRIEF DESCRIPTION OF THE DRAWINGS

FIG. 1 is chromatogram of an enzymatic cleavage of the albumin-bound form of compound 1 by PSA. Cleavage study with the albumin-bound form of compound 1 after incubation with human PSA (50 μg/mL) at 37° C. The concentration of anthracycline was 200 μM. HPLC: BioLogic Duo-Flow System from Biorad, Munich; Lambda 1000 Monitor from Bischoff (λ=495 nm); UV detection at 254 nm; column: Waters, 300 Å, Symmetry C18 [4,6×250 mm] with inlet column; flow rate: 1.2 mL/min; mobile phase A: 22% CH₃CN, 78% 4 mM sodium acetate (pH 5.0); mobile phase B: CH₃CN; gradient: 0-25 min with 100% mobile phase A; in 25-40 min to 70% CH₃CN, 30% 4 mM sodium acetate; 40-50 min with 70% CH₃CN, 30% 4 mM sodium acetate; and 50-60 min with 100% mobile phase A. Injection volume: 50 μL.

FIG. 2 is chromatogram of a cleavage study of the doxorubicin-dipeptide Doxo-Arg-Ser with PSA-positive prostate carcinoma CWR22. Chromatogram of an incubation study of the cleaved doxorubicin-dipeptide [Doxo-Arg-Ser] with CWR22 homogenate recorded at 37° C. after 0 hours, 6 hours, and 20 hours. The concentration of anthracycline was 125 μM. HPLC conditions: see FIG. 1. The CWR22 homogenate (250 mg/l mL) was produced by homogenization of CWR22 xenograft tumors in 50 mM tris-HCl buffer at pH 7.4 containing 1 mM monothioglycerol.

FIG. 3 is chromatogram of an enzymatic cleavage of the albumin-bound form of compound 2 by PSA. Chromatogram of an enzymatic cleavage of the albumin conjugate of compound 1 with prostate-specific antigen (20 μg/mL) after 30 minutes, 3, 9 and 24 hours, at 37° C. (pH 7.4). The concentration of the anthracycline was 50 μM. HPLC: BioLogic Duo-Flow System from Biorad, Munich; Lambda 1000 Monitor from Bischoff (λ=495 nm); UV detection at 254 nm. Column: Waters, 300 Å, Symmetry C18 [4.6×250 mm] with inlet column; flow rate: 1.2 mL/min; mobile phase A: 27.5% CH₃CN, 72.5% 20 mM potassium phosphate (pH 7.0); mobile phase B: CH₃CN; gradient: 0-25 min with 100% mobile phase A; in 25-40 min to 70% CH₃CN, 30% 20 mM potassium phosphate; 40-50 min with 70% CH₃CN, 30% 20 mM potassium phosphate; and 50-60 min with 100% mobile phase A. Injection volume: 50 μL.

FIG. 4 is chromatogram of an incubation study of compound 3 with human plasma at 37° C. after 5 and 90 minutes. Chromatogram of an incubation study of compound 3 with human plasma at 37° C. after 5 and 90 minutes. The concentration of the anthracycline was 100 μM. HPLC chromatography conditions: see FIG. 3.

FIG. 5 is chromatogram of an enzymatic cleavage of the albumin-bound form of compound 3 by PSA. Chromatogram of an enzymatic cleavage of the albumin conjugate of compound 3 with prostate-specific antigen (20 μg/mL) after 5 minutes, 3 hours, and 7 hours at 37° C. (pH 7.4). The concentration of the anthracycline was 100 μM. HPLC chromatography conditions: see FIG. 3.

FIG. 6 is graph of the tumor growth of the CWR22 xenograft model treated with compound 3.

FIG. 7 is graph of the tumor growth of the CWR22 xenograft model treated with compound 1.

### DETAILED DESCRIPTION OF THE INVENTION

The anthracycline component with an antitumoral effect is an active ingredient of the general formula:

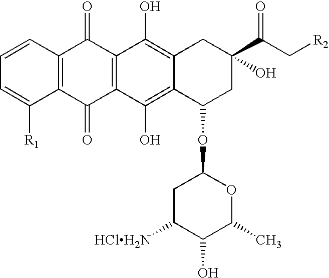

where

R₁═H, OH, or OCH₃; and

R₂═H or OH.

Preferred active ingredients are doxorubicin, daunorubicin, 4′-epirubicin, idarubicin, and carubicin.

An especially preferred active ingredient is doxorubicin.

The heterobifunctional crosslinker is a carboxylic acid derivative with a protein-binding group of the general formula:

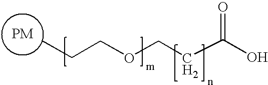

where

m=0-5;

n=0-6; and

PM=a protein-binding group.

It is preferable to use heterobifunctional crosslinkers with n<2 and m=2-5 and

n=4 and m=0. The oxyethylene units guarantee elevated solubility in water, especially at the larger values for m.

Values of n=4 and m=0 are especially preferred.

The protein-binding group (PM) is preferably selected from a 2-dithiopyridyl group, a halogen acetamide group, a halogen acetate group, a disulfide group, an acrylic acid ester group, a monoalkyl maleic acid ester group, a monoalkyl maleaminic acid amide group, a N-hydroxysuccinimidyl ester group, an isothiocyanate group, an aziridine group, or a maleinimide group. The maleinimide group is an especially preferred protein-binding group.

The peptide spacer is a peptide sequence P₁-P₁₀ consisting of L- and/or D-amino acids which are cleaved by PSA, where P₁ can be the amino acid Arg, His, Met, Ser, Tyr, Phe, Thr, Gly, Gln, or Lys. The preferred amino acid for P₁ is Arg. Preferred amino acids for P₁, P₂, P₃, P₄, P₅, P₆ are Ser, Tyr, Thr, Gln, Gly, Asn, and Phe, most preferably Ser and Tyr.

Preferred peptide spacers consist of six, seven, or eight amino acids.

Preferred sequences are:

Peptide Sequence

| P₁₀ | P₉ | P₈ | P₇ | P₆ | P₅ | P₄ | P₃ | P₂ | P₁ |
|---|---|---|---|---|---|---|---|---|---|
| | | | | Asn | Ser | Ser | Tyr | Phe | Gln |
| | | | | Ser | Ser | Tyr | Tyr | Ser | Gly |
| | | Arg | Arg | Ser | Ser | Tyr | Tyr | Ser | Gly |
| | | | Arg | Ser | Ser | Tyr | Tyr | Ser | Arg |
| | | | Arg | Ser | Ser | Tyr | Tyr | Gln | Arg |
| | | | Arg | Ser | Ser | Tyr | Tyr | Tyr | Arg |

An especially preferred sequence is Arg-Ser-Ser-Tyr-Tyr-Ser-Arg.

The inventive anthracycline peptide derivatives are produced effectively by reacting anthracycline active ingredients such as doxorubicin, daunorubicin, epirubicin, carubicin, or idarubicin with a peptide derivative of the general formula:

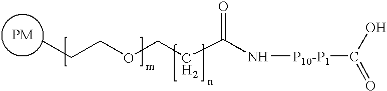

where

m=0-5;

n=0-6;

P₁-P₁₀ is a peptide sequence consisting of L- and/or D-amino acids; and

PM is a protein-binding group, by condensation of the activated carboxyl group of P₁ of the peptide derivative with the amino group of the active ingredient.

The inventive anthracycline peptide derivatives can also be produced effectively by reacting an anthracycline dipeptide of the general formula:

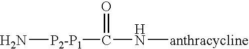

with a peptide derivative of the general formula:

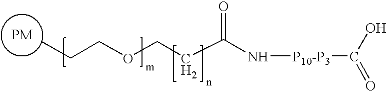

where

m=0-5;

n=0-6;

P₃-P₁₀ is a peptide sequence consisting of L- and/or D-amino acids; and

PM is a protein-binding group, by condensation of the activated carboxyl group of P₃ of the peptide derivative with the amino group of the anthracycline dipeptide.

The inventive anthracycline peptide derivatives can also be produced effectively by reacting an anthracycline-amino acid derivative of the general formula:

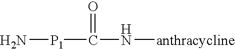

with a peptide derivative of the general formula:

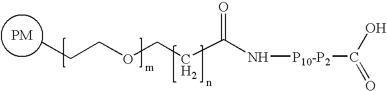

where

m=0-5;

n=0-6;

P₂—P₁₀ is a peptide sequence consisting of L- and/or D-amino acids; and

PM is a protein-binding group, by condensation of the activated carboxyl group of P₂ of the peptide derivative with the amino group of the anthracycline-amino acid derivative.

To activate the C-terminal end of the peptide derivative, it is preferable to use N,N′-dicyclohexylcarbodiimide (DCC), N,N′-diisopropylcarbodiimide (DIPC), (benzotriazol-1-yloxy)tris(dimethylamino)phosphonium hexafluorophosphate (BOP), N-[(dimethylamino)-1H-1,2,3-triazolo[4,5-b]pyridino-1-ylmethylene]-N-methylmethanaminium hexafluorophosphate (HATU), or 2-chloro-1-methylpyridinium iodide with the addition of standard catalysts or auxiliary bases such as trialkylamines, pyridine, 4-dimethylaminopyridine (DMAP), or hydroxybenzotriazole (HOBt). The reactions are conducted effectively in polar aprotic solvents such as DMF, DMA, or DMSO at temperatures between −20° C. and 40° C., preferably at 0-5° C., where the reaction time will usually be between 1 and 120 hours, preferably between 24 and 96 hours. The product can be isolated by crystallization, chromatography on silica gel, reversed-phase chromatography, or size-exclusion chromatography.

The inventive protein-binding anthracycline-peptide derivatives are administered parenterally, preferably intravenously. For this purpose, the inventive anthracycline-peptide derivatives are made available as solutions, solids, or lyophilisates, optionally with the use of conventional auxiliary materials. Such auxiliary materials include, for example, polysorbates, glucose, lactose, mannitol, sucrose, dextrans, citric acid, tromethamol, triethanolamine, aminoacetic acid, and/or synthetic polymers. The inventive anthracycline-peptide derivatives are preferably dissolved and administered in an isotonic buffer in a pH range of 2.0-8.0, preferably of pH 5.0-7.0. As a rule, the inventive anthracycline-peptide derivatives have adequate solubility in water because of the oxyethylene units in the crosslinker and/or because of the integration of polar amino acids such as Arg, His, Ser, Tyr, or Lys into the peptide sequence. The solubility of the anthracycline-peptide derivative can be improved, if desired, by the addition of pharmaceutical solvents such as 1,2-propanediol, ethanol, isopropanol, glycerol, and/or polyethylene glycol with a molecular weight of 200-600 g/mol, preferably polyethylene glycol with a molecular weight of 600 g/mmol, and/or solubilizers such as Tween 80, Cremophor, or polyvinylpyrrolidone.

An essential feature of the inventive anthracycline-peptide derivatives is their rapid covalent binding to serum proteins via the protein-binding group, as a result of which a macromolecular transport from of the active ingredient is generated. It is known that tumor tissue takes up increased amounts of serum proteins such as transferrin and albumin (Kratz F., Beyer U: Drug Delivery, 1998, 5, 281-299), which means that they can be used within the scope of the invention as so-called “endogenous carriers” for cytostatics. An especially preferred serum protein is circulating human serum albumin (HSA), which has an average concentration of 30-50 g/L and is thus the primary protein component of human blood (Peters T, Advantage. Protein Chem., 1985, 37, 161-245). A free cysteine group (cysteine-334 group), which is suitable as an attachment site for the binding of thiol-binding groups such as maleinimides and disulfides (WO 00/76551), is present on the surface of this protein. The reaction of the anthracycline-peptide derivative with serum proteins can also be conducted extracorporeally, e.g., with a quantity of albumin, blood, or serum intended for infusion.

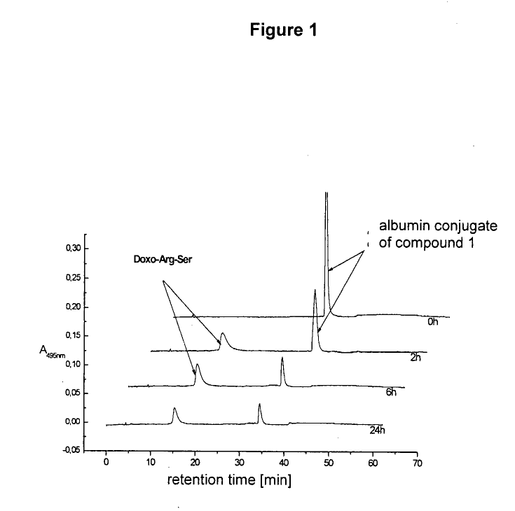

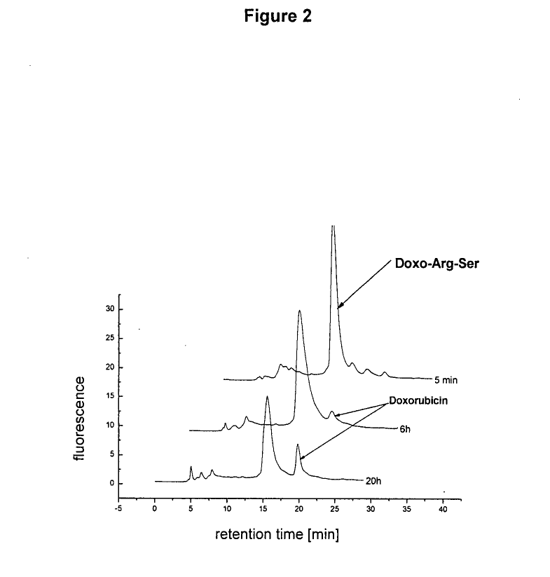

The biodistribution of protein-bound anthracycline-peptide derivatives is different from that of the free active ingredient. As a result of their macromolecular character, they accumulate in tumor tissue, and when they are cleaved by PSA in the tumor tissue, low-molecular anthracycline-peptides are released, which are then able to exert an antitumoral effect (see FIGS. 1, 3, 4, and 5). The anthracycline peptide can also be cleaved back to the original anthracycline in tumor tissue (see FIG. 2). In animal experiments, protein-binding doxorubicin-peptide derivatives showed very good antitumoral efficacy (see Example 1).

The following examples will explain the invention in greater detail in conjunction with the figures.

### EXAMPLE 1

### Synthesis of EMC-Arg-Ser-Ser-Tyr-Tyr-Ser-Arg-Doxo (see Compound 1) with Doxo-Arg-Ser

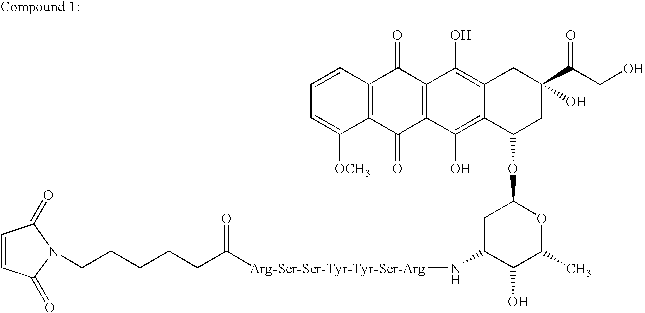

### 1. Synthesis of Doxorubicin-Arg:

200 mg (0.3448 mmol) of doxorubicin hydrochloride, 305 mg (0.77 mmol) of Fmoc-Arg-OH, and 0.00031 mg, 200 μL (31.5 mmol), of triethylamine were dissolved in 25 mL of dry DMF. The solution was stirred at 25° C. (RT) for 5 minutes, and then 157.3 mg (0.4138 mmol, 1.2 equivalents) of HATU was added as a coupling reagent. Then the mixture was stirred at 25° C. (RT) for 2 hours. The product was precipitated with 1,000 mL of diethyl ether; the precipitate was washed three times with diethyl ether and dried under vacuum. The Fmoc protective group was removed by treating the sample with 5 mL of a 20% piperidine solution in DMF. After a reaction time of 5 minutes, the product was precipitated with 250 mL of diethyl ether and washed three times with 20 mL of ether. The product was then purified on a diol column, i.e., LiChroprep DIOL (40-63 μm), with the use of chloroform/methanol 3:1+0.1% TFA, chloroform/methanol 2:1+0.1% TFA, and methanol+0.1% TFA in that order as mobile phases. (The sample must first be combined with 0.5% TFA so that it is soluble in the mobile phase.) The fractions containing the product were collected, precipitated with diethyl ether, and dried under high vacuum, as a result of which 322.5 mg of the target compound was obtained as a red powder. Mass (ESI: 2.5 kV, Mr 699.7): m/z 700.2 [M+H]⁺, HPLC (495 nm): >98%.

### 2. Synthesis of Doxorubicin-Arg-Serg:

390 mg (0.558 mmol) of Doxo-Arg-OH, 390.52 mg (1.193 mmol) of Fmoc-Ser-OH, and 49.97 mg, 426 μL (2.512 mmol), of DIEA were dissolved in 22 mL of dry DMF and stirred at 25° C. (RT) for 5 minutes. 318.24 mg (0.837 mmol) of HATU was added as a coupling reagent, and the solution was stirred at 25° C. for 2 hours. The product was then precipitated with 1,000 mL of diethyl ether, and the precipitate thus obtained was washed three times with 20 mL of diethyl ether and dried under vacuum. After purification of the product by column chromatography (chloroform/methanol 5:1+0.1% trifluoroacetic acid), the protective group was removed by treating the sample with a 20% piperidine solution in DMF. After a reaction time of 5 minutes, the product was precipitated with 50 times the amount of diethyl ether and washed three times with ether. The precipitate was then dried under high vacuum, as a result of which 186.3 mg of Doxo-Arg-Ser was obtained. Mass (ESI: 3 kV, Mr 787.2): m/z 788.2 [M+H]⁺, HPLC (495 nm): >95%.

### 3. Synthesis of EMC-Arg-Ser-Ser-Tyr-Tyr-Ser-Arg-Doxo (see Compound 1)

128 mg (0.163 mmol) of doxorubicin-Arg-Ser, 159.33 mg (0.184 mmol) of EMC-Arg-Ser-Ser-Tyr-Tyr-OH (EMC=maleinimidocaproic acid), 65.87 mg (0.487 mmol) of 1-hydroxybenzotriazole hydrate, and 70.86 μL (65.21 mg, 0.643 mmol) of 4-methylmorpholine in 10 mL of dry N,N-dimethylformamide (DMF) were stirred at +5° C. for 15 minutes. 150.68 μL (123.07 mg, 0.975 mmol) of N,N′-diisopropylcarbodiimide was added, and the mixture was stirred at +5° C. for 72 hours. Then the product was precipitated with 50 times the amount of diethyl ether (500 mL), and the supernatant diethyl ether was decanted. The precipitate was washed with 3×20 mL of diethyl ether and dried under vacuum. The product was dissolved in MeOH/water 3:1 and purified by two size-exclusion chromatography runs on Sephadex™ LH-20 (Amersham Pharmacia Biotech AB) with methanol. The solvent was removed from the obtained fractions under vacuum, after which lyophilization was carried out with acetonitrile/water 50:50 under high vacuum. As a result, 152 mg of compound 1 was obtained as a red powder. Mass (LC-MS-pos. ESI. 1.5 kV, Mr 1636.5): m/z 1637.5 [M+H]⁺, 1749.7 [M⁺+CF₃COO⁻], 1750.7 [M⁺+CF₃COO⁻H⁺], HPLC (495 nm): >95%.

### EXAMPLE 2

### Enzymatic Cleavage of the Albumin-Bound Form of Compound 1 by PSA

### Production of the Albumin Conjugate of Compound 1

1.8 mg of compound 1 was incubated with 1 mL of commercial human serum albumin at 37° C. for 1 hour. The resulting albumin conjugate was purified by size-exclusion chromatography (Sephacryl® HR100; buffer 0.004 M sodium phosphate, 0.15 M sodium chloride; pH 6.5).

The albumin-bound form of compound 1 [200 μM] was incubated with human prostate-specific antigen (50 μg/mL) at 37° C. and detected by chromatography on a C₁₈-RP-HPLC column (Symmetry® 300-5 4.6×250 mm from Waters) by gradient elution (flow rate: 1.2-1.8 mL/min; eluent A: 22% acetonitrile, 78% 4 mmol sodium acetate buffer at pH 5.0; eluent B: 30% 4 mmol sodium acetate buffer at pH 5.0, 70% acetonitrile; gradient: 0-25 min with 100% mobile phase A; in 25-40 min to 70% acetonitrile, 30% 4 mM sodium acetate; 40-50 min with 70% CH₃CN, 30% 4 mM sodium acetate; 50-60 min with 100% mobile phase A) at the times shown in FIG. 1 at 495 nm. Injection volume: 50 μL.

Incubation studies, which were conducted with the albumin conjugate of compound 1 and human PSA (Calbiochem, FRG) confirmed that the doxorubicin-dipeptide Doxo-Arg-Ser was released (FIG. 1).

This is cleaved in tumor tissue (CWR22 tissue homogenate) to doxorubicin (see FIG. 2).

### EXAMPLE 3

### Cleavage Study of the Doxorubicin-Dipeptide Doxo-Arg-Ser with PSA-Positive Prostate Carcinoma CWR22

An incubation study at 37° C. with CWR22 tissue homogenate at pH 7.4 was conducted with the doxorubicin dipeptide (Doxo-Arg-Ser) obtained as described in Example 2. The concentration of anthracycline was 100 μM. HPLC chromatography was conducted under the conditions of Example 2 after 5 minutes, 6 hours, and 20 hours. The results obtained are shown in FIG. 2. The cleavage study confirmed the interesting fact that the doxorubicin dipeptide (Doxo-Art-Ser) cleaved by PSA is cleaved to doxorubicin in tumor tissue (CRW22).

### EXAMPLE 4

### Synthesis of Mal-Asn-Ser-Ser-Tyr-Phe-GIn-Doxo (PSA3) (see Compound 2)

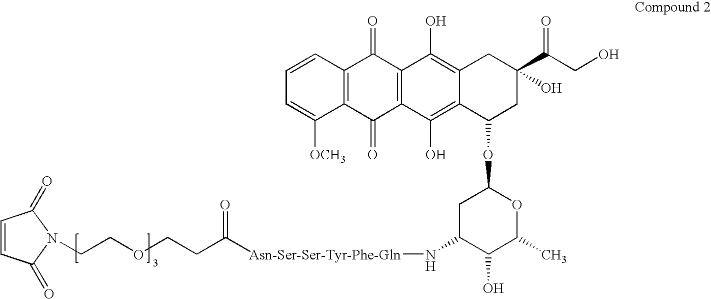

58 mg (0.1 mmol) of doxorubicin hydrochloride, 102.8 mg (0.1 mmol) of Mal-Asn-Ser-Ser-Tyr-Phe-Gln-OH (Mal=maleinimidotriethylenglycolic acid), 13.5 mg (0.1 mmol) of 1-hydroxybenzotriazole hydrate, and 33 μL (30.3 mg, 0.3 mmol) of 4-methylmorpholine were stirred in 20 mL of dry N,N-dimethylformamide (DMF) at +5° C. for 15 minutes. 46.5 μL (37.9 mg, 0.3 mmol) of N,N′-diisopropylcarbodiimide was added, and the mixture was stirred at +5° C. for 96 hours. Then the DMF was removed under high vacuum. The residue was dissolved in chloroform/methanol 3/1 and purified by two column chromatography runs on silica gel 60 (Merck, Darmstadt) with chloroform/methanol 3/1. 50 mg of compound 2 was obtained as a red powder. Mass (ESI-MS, Mr 1553.5): m/z 1576 [M+Na]⁺, HPLC (495 nm): >98%.

### EXAMPLE 5

### Enzymatic Cleavage of the Albumin-Bound Form of Compound 2 by PSA Production of the Albumin Conjugate of Compound 2

12.1 mg of compound 2 was incubated with 10 mL of commercial human serum albumin for 1 hour at 37° C. The resulting albumin conjugate was purified by size-exclusion chromatography (Sephacryl® HR1001; buffer, 0.004 M sodium phosphate, 0.15 M sodium chloride pH 6.5).

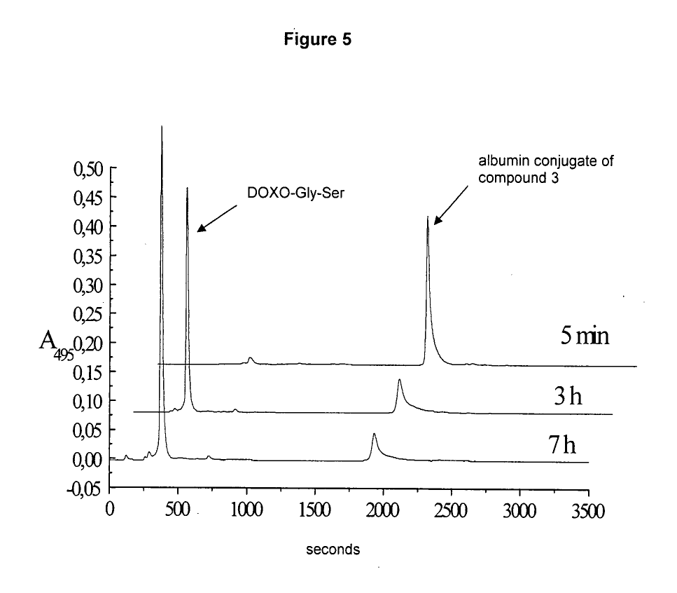

The albumin-bound form of compound 2 [200 μM] was incubated with human prostate-specific antigen (20 μg/mL) at 37° C. and detected by chromatography on a C₁₈-RP-HPLC column (Symmetry® 300-5 4.6×250 mm from Waters) by gradient elution (flow rate: 1.2 mL/min, mobile phase A: 27.5% CH₃CN, 72.5% 20 mM potassium phosphate (pH 7.0); mobile phase B: CH₃CN; gradient: 0-25 min with 100% mobile phase A; in 25-40 min to 70% CH₃CN, 30% 20 mM potassium phosphate; 40-50 min with 70% CH₃CN, 30% 20 mM potassium phosphate; 50-60 min with 100% mobile phase A) at the times shown in FIG. 5 at 495 nm. Injection volume: 50 μL.

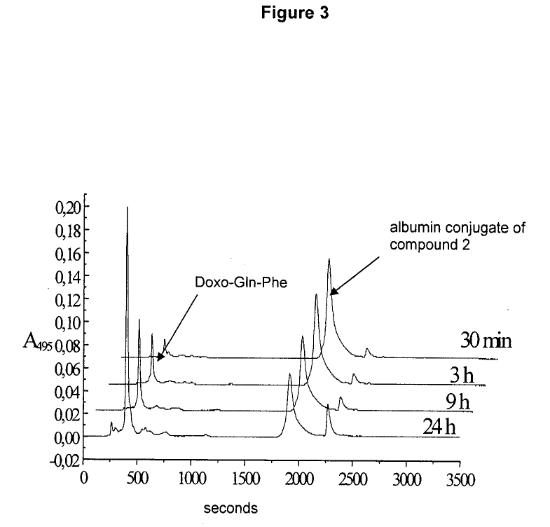

Incubation studies conducted with the albumin conjugate of compound 2 and human PSA (Calbiochem, FRG) confirmed that the doxorubicin dipeptide Gln-Phe-Doxo was released (FIG. 3).

### EXAMPLE 6

### Synthesis of EMC-Arg-Arg-Ser-Ser-Tyr-Tyr-Ser-Gly-Doxo (see Compound 3)

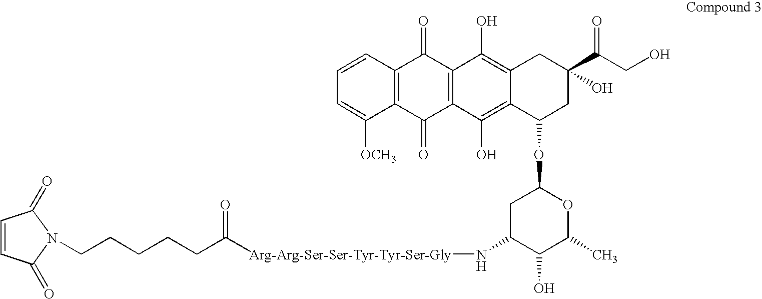

50 mg (0.086 mmol) of doxorubicin hydrochloride, 100.7 mg (0.086 mmol) of EMC-Arg-Arg-Ser-Ser-Tyr-Tyr-Ser-Gly-OH (EMC=maleinimidocaproic acid), 11.6 mg (0.086 mmol) of 1-hydroxybenzotriazole hydrate, and 37.8 μL (37.8 mg, 0.34 mmol) of 4-methylmorpholine in 20 mL of dry N,N-dimethylformamide (DMF) were stirred for 15 minutes at +5° C. 39.8 μL (32.5 mg, 0.26 mmol) of N,N′-diisopropylcarbodiimide was added, and the mixture was stirred for 96 hours+5° C. Then the product was precipitated with diethyl ether and washed 3 times with 20 mL of diethyl ether. 130 mg of compound 3 was obtained as a red powder. Mass (ESI-MS, Mr 1693.7): m/z 1807.6 [M+Na]⁺, HPLC (495 nm): >97%.

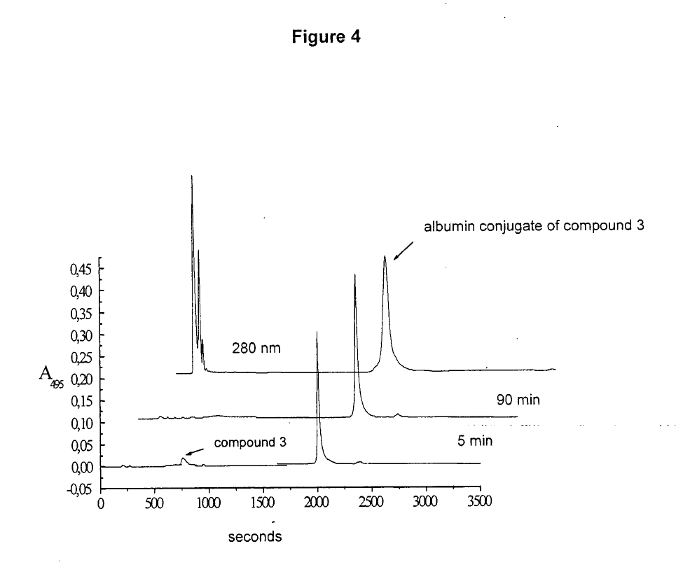

Compound 3 binds selectively within a few minutes to the cysteine-34 position of endogenous albumin in blood plasma (see FIG. 4).

### EXAMPLE 7

### Enzymatic Cleavage of the Albumin-Bound Form of Compound 3 by PSA

The albumin-bound form of compound 3 [200 μM] was incubated with human prostate-specific antigen (20 μg/mL) at 37° C. and detected by HPLC chromatography under the conditions of Example 3 at the times indicated in FIG. 5 at 495 nm. Injection volume: 50 μL.

Incubation studies conducted with the albumin conjugate of compound 3 and human PSA (Calbiochem, FRG) confirmed that the doxorubicin dipeptide Doxo-Gly-Ser was released (FIG. 5).

### EXAMPLE 8

### In-vivo Activity of Compound 3 in the PSA-positive Xenograft Model (CWR22)

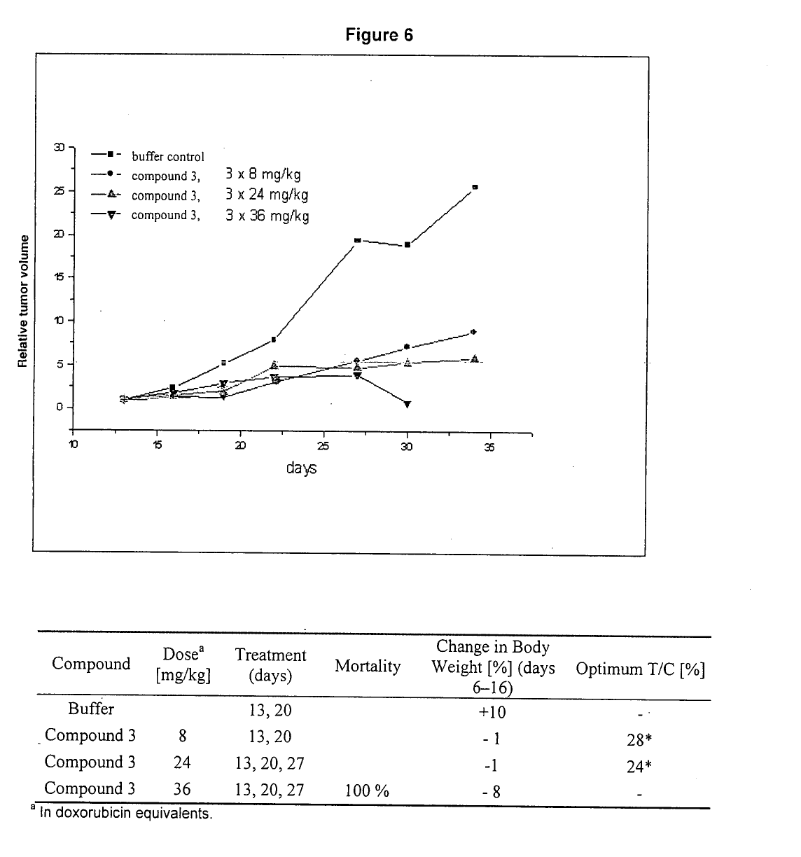

The course of the tumor growth of the subcutaneously growing PSA-positive xenograft model CWR22, which was treated with structure 3 [dose (i.v.): 2×13.3 μmol/kg (=2×8 mg/kg doxorubicin equivalents) on days 13 and 20; 3×39.9 μmol/kg (=3×24 mg/kg doxorubicin equivalents) on days 13, 20, and 27; and 3×59.9 μmol/kg (=3×36 mg/kg doxorubicin equivalents) on days 13, 20, and 27] is shown in FIG. 6.

The figure shows the relative tumor volumes at the indicated times.

Animals: hairless mice. Stock solution of compound 3:6.0 mg/mL in 10 mM sodium phosphate, 5% D-glucose (pH 6.4); control (buffer): glucose-phosphate buffer (10 mM sodium phosphate, 5% D-glucose—pH 6.4) on days 13 and 20.

The curves in FIG. 6 confirm that compound 3 has a good antitumoral effect.

### EXAMPLE 9

### In-vivo Activity of Compound 1 in the PSA-positive Xenograft Model (CWR22)

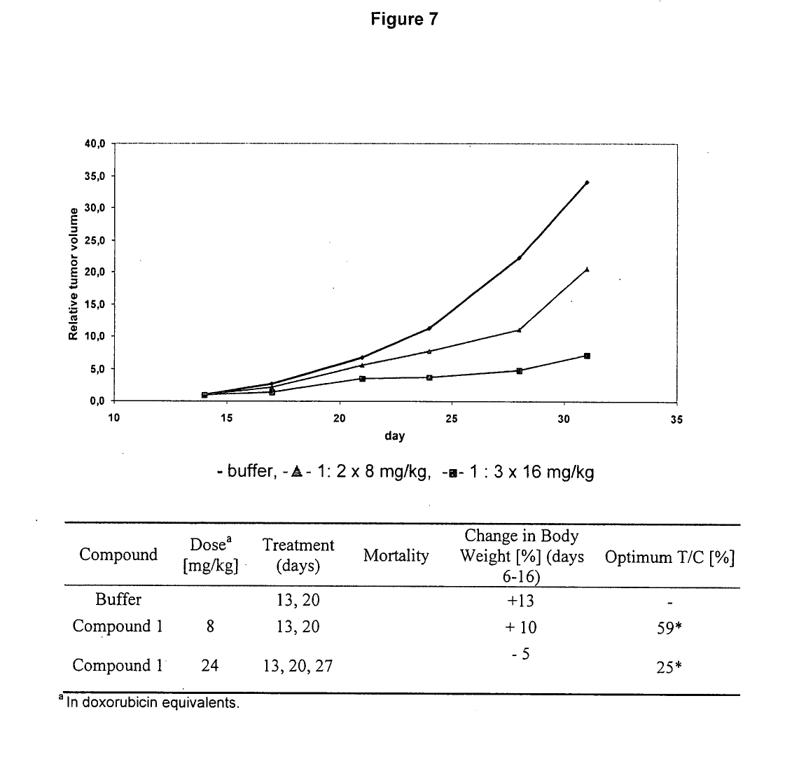

The course of the tumor growth of the subcutaneously growing PSA-positive xenograft model CWR22, which was treated with structure 1 [dose (i.v.): 2×13.3 μmol/kg (=2×8 mg/kg doxorubicin equivalents) on days 13 and 20; 3×26.3 μmol/kg (=3×16 mg/kg doxorubicin equivalents) or with buffer (control) on days 13 and 20], is shown in FIG. 7.

The figure shows the relative tumor volumes at the indicated times.

Animals: hairless mice. Stock solution of compound 1:7.4 mg/mL in 10 mM sodium phosphate, 5% D-glucose (pH 6.4); control (buffer): glucose-phosphate buffer (10 mM sodium phosphate, 5% D-glucose—pH 6.4) on days 13 and 20.

The curves in FIG. 7 confirm that compound 3 has a good antitumoral effect.

1. An anthracycline-peptide derivative of Formula I: 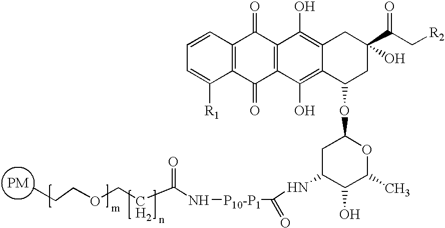
wherein
R₁═H, OH, or OCH₃;
R₂═H or OH;
m=0-5;
n=0-6;
P₁-P₁₀ is a peptide sequence cleavable by PSA consisting of L- and/or D-amino acids; and
PM is protein-binding group.

2. The anthracycline-peptide derivative according to claim 1, wherein the anthracycline component is doxorubicin, daunorubicin, 4′-epirubicin, idarubicin, or carubicin.

3. The anthracycline-peptide derivative according to claim 1, wherein the anthracycline component is doxorubicin.

4. The anthracycline-peptide derivative according to claim 1, wherein PM is a maleinimide group, a 2-dithiopyridyl group, a halogen acetamide group, a halogen acetate group, a disulfide group, an acrylic acid ester group, a monoalkyl maleic acid ester group, a monoalkyl maleaminic acid amide group, a N-hydroxysuccinimidyl ester group, an isothiocyanate group, or an aziridine group, which can optionally be substituted.

5. The anthracycline-peptide derivative according to claim 2, wherein PM is a maleinimide group, which can optionally be substituted.

6. The anthracycline-peptide derivative according to claim 1, wherein n<2 and m=2-5.

7. The anthracycline-peptide derivative according to claim 1, wherein n=4 and m=0.

8. The anthracycline-peptide derivative according to claim 1, wherein P₁ in the peptide sequence (P₁-P₁₀) is Arg, His, Met, Ser, Tyr, Thr, Phe, Gly, GIn, or Lys.

9. The anthracycline-peptide derivative according to claim 7, wherein P₁ in the peptide sequence is Arg.

10. The anthracycline-peptide derivative according to claim 1, wherein P₁, P₂, P₃, P₄, P₅, and P₆ in the peptide sequence are the same or different and stand for Ser, Tyr, Thr, Asn, Gln, Gly, or Phe.

11. The anthracycline-peptide derivative according to claim 1, wherein P₁, P₂, P₃, P₄, P₅, and P₆ in the peptide sequence are the same or different and stand for Ser or Tyr.

12. The anthracycline-peptide derivative according to claim 1, wherein the peptide sequence P₁-P₁₀ comprises six, seven, or eight amino acids.

13. The anthracycline-peptide derivative according to claim 1, wherein the peptide sequence is Arg-Ser-Ser-Tyr-Tyr-Ser-Arg, Arg-Arg-Ser-Ser-Tyr-Tyr-Ser-Gly, Ser-Ser-Tyr-Tyr-Ser-Gly, Asn-Ser-Ser-Tyr-Phe-Gln, Arg-Ser-Ser-Tyr-Tyr-Gln-Arg, or Arg-Ser-Ser-Tyr-Tyr-Tyr-Arg.

14. The anthracycline-peptide derivative according to claim 1, wherein the peptide sequence is Arg-Ser-Ser-Tyr-Tyr-Ser-Arg.

15. A process for the production of a doxorubicin-peptide derivative according to claim 1, the process comprising reacting doxorubicin with a peptide derivative of General Formula II: 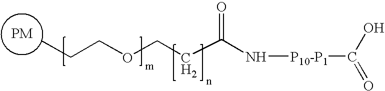
wherein
m=0-5;
n=0-6;
P₁-P₁₀ is a peptide sequence consisting of L- and/or D-amino acids; and
PM is a protein-binding group,
in the presence of carboxylic acid activation reagents.

16. The process according to claim 15, wherein PM is a maleinimide group.

17. The process for the production of doxorubicin-peptide derivatives, said process comprising reacting doxorubicin with a peptide of General Formula III: 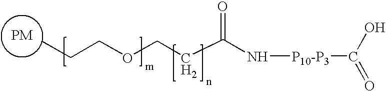
wherein
m=0-5;
n=0-6;
P₃-P₁₀ is a peptide sequence, consisting of L- and/or D-amino acids; and
PM is a protein-binding group,
in the presence of carboxylic acid activation reagents.

18. The process according to claim 17, wherein PM is a maleinimide group.

19. The process according to any one of-claims 15 or 17, wherein the carboxylic acid activation reagent is selected from N,N′-dicyclohexylcarbodiimide, N,N′-diisopropylcarbodiimide, N-[(dimethylamino)-1H-1,2,3-triazolo[4,5-b]pyridino-1-ylmethylene]-N-methylmethanaminium hexafluorophosphate (HATU), or (benzotriazol-1-yloxy)tris(dimethylamino)phosphonium hexafluorophosphate, preferably N,N-diisopropylcarbodiimide.

20. A composition comprising the anthracycline-peptide derivative of claim 1, optionally together with pharmaceutical auxiliary substances and/or solvents.

21. A method for the treatment of cancer diseases comprising administering to a subject in need of treatment of cancer disease a composition comprising the anthracycline-peptide derivative of claim 1.

22. A process for the production of a composition, the process comprising converting a compound of claim 1 into a therapeutically compatible solution.
